(19) United States
(12) Patent Application Publication
(10) Pub. No.: US 2023/0287669 A1
(43) Pub. Date: Sep. 14, 2023

(54) WASHING AND DRYING NOZZLE DEVICE FOR SANITARY TOILET SEAT COVER

(71) Applicant: Bum Suk YOUN, Gangneung-si (KR)

(72) Inventor: Bum Suk YOUN, Gangneung-si (KR)

(21) Appl. No.: 18/061,537

(22) Filed: Dec. 5, 2022

(30) Foreign Application Priority Data
Mar. 11, 2022 (KR) 10-2022-0030787

(51) Int. Cl. E03D 9/08; A47K 10/48; E03D 9/00; A47K 13/30

(52) U.S. Cl. CPC E03D 9/08; A47K 10/48; E03D 9/002; A47K 13/302

(57) ABSTRACT

Disclosed herein is a washing and drying nozzle device for a sanitary toilet seat cover. The washing and drying nozzle device includes: a toilet seat-cover device including a support frame configured such that a cover member and a seat are rotatably coupled thereto and a sensor unit configured to detect the presence of a foreign material is mounted on the front portion thereof; a drying nozzle coupled to the support frame, and configured to dry a user's washed body by spraying air toward the user's body and to radiate far-infrared rays toward the user's body; a washing nozzle coupled to the support frame, and configured to spray water toward the user's body; a nozzle driving means installed in the support frame, and configured to slide and rotate the drying nozzle and the washing nozzle; and a control unit configured to control the driving of the nozzle driving means.

### CROSS-REFERENCE TO RELATED APPLICATION

This application claims the benefit of Korean Patent Application No. 10-2022-003078 filed on 2022 Mar. 11, which is hereby incorporated by reference herein in its entirety.

### BACKGROUND

### 1. Technical Field

The present invention relates generally to a washing and drying nozzle device for a sanitary toilet seat cover, and more particularly to a washing and drying nozzle device for a sanitary toilet seat cover that radiates far-infrared rays directly to a user's body, so that drying efficiency can be improved after washing with water, and so that a sitz bath function and a sterilization function can be provided by the efficacy of far-infrared rays, thereby enabling sanitary management after the defecation of the user and health management for a specific body part.

### 2. Description of the Related Art

In general, a bidet is a toilet bowl that deals with washing, bidet, and drying processes after defecation. Bidets are widely distributed in toilets in homes and public places, and can perform washing, bidet, and drying operations in various modes in response to the manual operation of a user. A user can control the washing, bidet, and drying operations of such a bidet by inputting and setting the strength of water pressure to be discharged from the bidet, the temperature of water, the temperature of a wind, and the temperature of a bidet seat.

However, in the case of the prior art bidet, when a user selects a washing operation after defecation, the washing water sprayed to the user's anus or genital area splashes the contaminants washed by the washing water into a toilet seat, so that there is a problem in that bacteria, E. coli, viruses, and/or the like harmful to the human body land on the inside of a toilet bowl and proliferate.

Accordingly, as disclosed in Korean Patent No. 10-2291716, the present applicant filed and obtained a patent for a sanitary toilet seat-cover device, including: a cover member configured such that a first sterilizing unit is mounted on the bottom surface thereof and a connection groove configured to support an electrical connection with the first sterilizing unit is formed in the front side thereof; a seat configured such that a plurality of seating portions are formed on the bottom surface thereof, a heating wire is embedded in the inside thereof, and a fixed magnet is mounted on the front side thereof; a grip part including an airtight housing inserted into the connection groove and configured to support a rotating operation, an operating member inserted into the sealed housing, configured to perform a rotating operation, and provided with a rotating magnet that is mounted at one end of the operating member so that the operating member is coupled to or separated from the fixed magnet by magnetic force, a grip body configured to rotate the operating member, a second sterilizing unit and battery mounted on the top surface of the grip body, and a grip cover coupled to the top portion of the grip body and configured such that ultraviolet rays radiated when the second sterilizing unit is driven are transmitted therethrough; and a washing part including a control unit configured to control the driving of the driving unit when receiving a control signal, a controller unit including a remote control configured to be connected to a wireless network and transmit a control signal to the control unit, a manipulation frame configured such that a button unit configured to generate and transmit the control signal is formed therein and the cover member and the seating seat are rotatably coupled thereto, a nozzle member configured to discharge air and water having a predetermined pressure and temperature, and a driving unit configured to drive the nozzle member under the control of the control unit and supply the air and water to the nozzle member.

However, in the case of the above-described patented invention, it is difficult to consider that drying efficiency for a user's body is excellent only based on the fact that the nozzle member simply sprays air and water having a predetermined pressure. In particular, when a foreign material is formed in the nozzle member itself, there is a concern that contaminated water may be sprayed toward the user's body or the foreign material may move toward a user's body due to the spray pressure of air. Therefore, there is a need to further maximize sanitation.

The above-described background art or prior art is only to help understand the technical significance of the present invention, and does not mean the technology that is widely known in the art to which this invention pertains prior to the filing of the present invention.

### PRIOR ART LITERATURE

Patent document: Korean Patent No. 10-2291716

### SUMMARY

The present invention has been conceived to overcome the above-described problems, and an object of the present invention is to provide a washing and drying nozzle device for a sanitary toilet seat cover that radiates far-infrared rays directly to a user's body, so that drying efficiency can be improved after washing with water, and so that a sitz bath function and a sterilization function can be provided by the efficacy of far-infrared rays, thereby enabling sanitary management after the defecation of the user and health management for a specific body part.

Another object of the present invention is to provide a washing and drying nozzle device for a sanitary toilet seat cover that radiates far-infrared rays toward a water pressure nozzle before, during, and after the use of a bidet, so that the water pressure nozzle can be sterilized regardless of whether the bidet is used, thereby enabling the sanitary management of the water pressure nozzle.

Still another object of the present invention is to provide a washing and drying nozzle device for a sanitary toilet seat cover that provides water and far-infrared rays to a user by using a water pressure nozzle and a drying nozzle, so that it is possible to dispose of defecation sanitarily even without using toilet paper, with the result that the present invention is eco-friendly, can improve toilet culture, and can be used conveniently by the elderly, patients, children, and the disabled.

However, the objects of the present invention are not limited thereto, and the objects that can be derived from the solutions to the problems or embodiments are also included therein even when they are not explicitly mentioned herein.

In order to accomplish the above objects, the present invention provides a washing and drying nozzle device for a sanitary toilet seat cover, the washing and drying nozzle device including: a toilet seat-cover device including a support frame configured such that a cover member and a seat are rotatably coupled thereto and a sensor unit configured to detect the presence of a foreign material is mounted on one side of the front portion thereof; a drying nozzle coupled to the support frame, and configured to dry a user's washed body by spraying air having a predetermined pressure toward the user's body and to radiate far-infrared rays toward the user's body; a washing nozzle coupled to the support frame, and configured to spray a predetermined amount of water toward the user's body; a nozzle driving means installed in the support frame, and configured to slide and rotate the drying nozzle and the washing nozzle; and a control unit configured to control the driving of the nozzle driving means.

According to an embodiment of the present invention, the control unit may receive detection result information about whether a foreign material is present on the surface of the washing nozzle from the sensor unit, and, when a foreign material is present, may drive the drying nozzle to radiate far-infrared rays toward the washing nozzle so that sterilization is achieved.

According to an embodiment of the present invention, the drying nozzle may include: an air inlet hole formed in one side of the rear portion of the drying nozzle, and configured such that air having a predetermined pressure is introduced therethrough; an air discharge hole formed in the top of the front side of the drying nozzle, and configured such that introduced air and far-infrared rays are discharged therethrough; a lamp assembly disposed below the air discharge hole, and configured to radiate far-infrared rays onto the user's body or the surface of the washing nozzle through the air discharge hole under the control of the control unit; an operation support flange disposed at the center of the rear side of the drying nozzle, and configured to perform support so that an electrical connection is made between the lamp assembly and the control unit and to support the sliding movement and rotation operation of the drying nozzle; a nozzle operation support means configured to connect the operating support flange and the nozzle driving means to each other, and to transmit the rotational force of the nozzle driving means to the operating support flange so that a rotating operation for up/down inversion of the drying nozzle can be performed; and an air supply member configured to supply a predetermined amount of air toward the air inlet hole.

According to an embodiment of the present invention, the washing nozzle may be independently disposed below the drying nozzle, or may be coupled to allow sliding movement inside the drying nozzle.

According to an embodiment of the present invention, the washing nozzle may further include a nozzle connection means configured to support the sliding movement and rotating operation of the washing nozzle and to supply a predetermined amount of water.

According to an embodiment of the present invention, the control unit may control the driving of the nozzle driving means so that water can be sprayed around a toilet bowl by rotating the washing nozzle over a predetermined angle in the left and right directions and then drying and sterilization can be performed by rotating the drying nozzle in the left and right directions.

According to an embodiment of the present invention, the control unit may perform control so that far-infrared rays can be radiated toward the user's body by operating the drying nozzle even while the user is defecating.

According to an embodiment of the present invention, the control unit may operate in conjunction with a remote control that provides a drive control signal, a control signal for whether to use hot water, a control signal for whether to radiate far-infrared rays, control signals for washing and drying operations, and a control signal for the user's physical condition.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a view schematically showing a state in which a washing and drying nozzle device for a sanitary toilet seat cover according to an embodiment of the present invention is mounted;

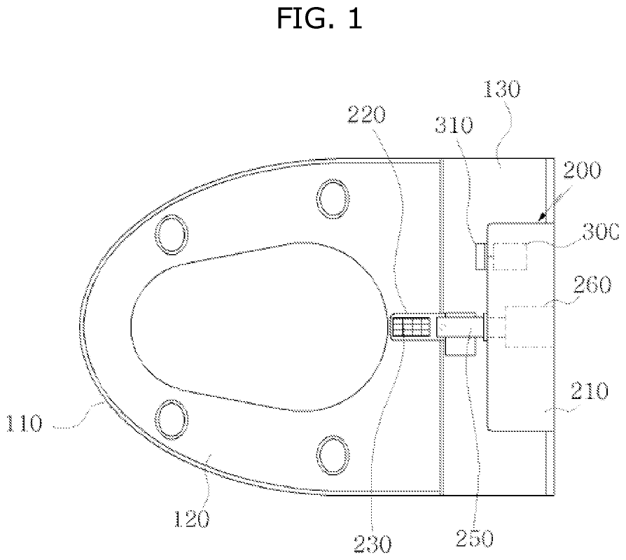

FIG. 2 is a perspective view showing a drying nozzle device according to an embodiment of the present invention;

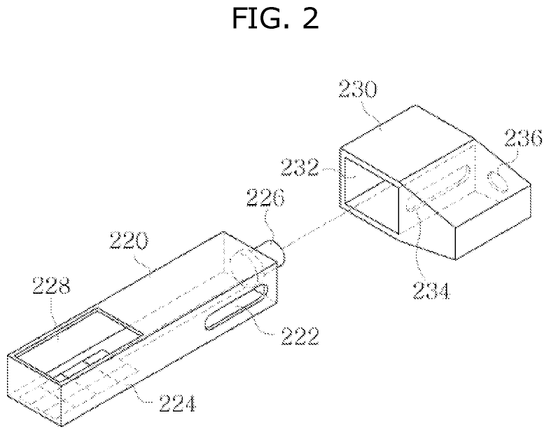

FIG. 3 is a cross-sectional view schematically showing the internal structure of the drying nozzle device according to the embodiment of the present invention; and

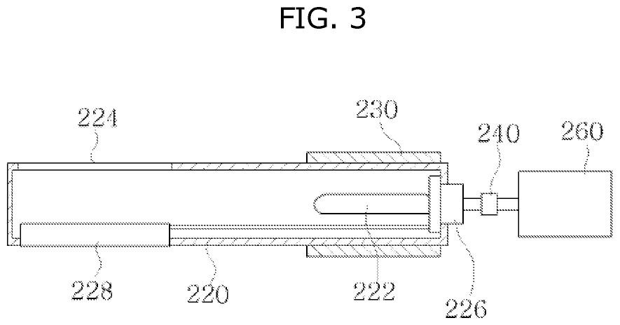

FIG. 4 is a view showing a washing and drying nozzle device for a sanitary toilet seat cover according to another embodiment of the present invention.

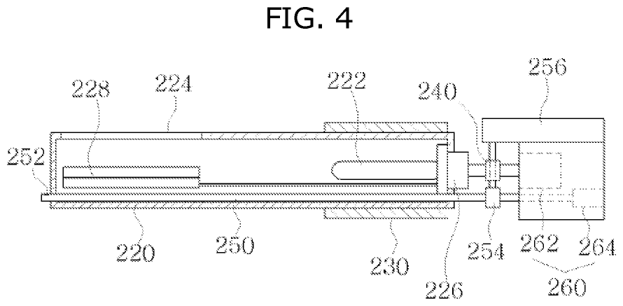

### DETAILED DESCRIPTION

Embodiments of the present invention will be described in detail below with reference to the accompanying drawings. First, it should be noted that when reference numerals are assigned to components of the drawings, the same components have the same numerals as much as possible even when they are shown on different drawings. Furthermore, in the following description of the present invention, when it is determined that a detailed description of a related known configuration or function may obscure the gist of the present invention, the detailed description will be omitted.

In addition, terms such as “include,” “comprise,” or “have” described below mean that a corresponding component may be inherent unless otherwise stated. It should be construed as possibly further including another component, rather than excluding another component. All terms, including technical or scientific terms, have the same meanings as commonly understood by a person of ordinary skill in the art to which the present invention pertains, unless defined otherwise. Also, terms such as first, second, A, B, (a), and (b) may be used in the description of the components of the present invention. These terms are only used to distinguish corresponding components from other components, and the natures, sequential positions and/or sequence of the corresponding components are not limited by the terms. When an element is described as being “connected,” “coupled,” or “attached” to another element, it will be understood that the former element may be directly connected, coupled or attached to the other element, but there may be another element between the elements.

Embodiments of the present invention will be described in detail below with reference to the accompanying drawings.

As shown in the drawings, a washing and drying nozzle device for a sanitary toilet seat cover according to the present invention includes a toilet seat-cover device 100, and a washing and drying nozzle device 200 coupled to the toilet seat-cover device 100, and configured to clean a user after defecation by spraying water and air to a part of the user's body, and to perform sterilization by radiating far-infrared rays onto nozzle devices and, at the same time, further maximize drying efficiency and also promote the user's health by radiating far-infrared rays onto a part of the user's body.

In this case, the toilet seat-cover device 100 includes a cover member 110 and a seat 120, is mounted with the washing and drying nozzle device 200, and further includes a support frame 130 configured to perform support such that the sliding movement and rotating operation of the washing and drying nozzle device 200 can be performed.

In this case, the cover member 110 and the seat 120 are rotatably coupled to the front portion of the support frame 130, and the support frame 130 is provided with cables such as signal and power lines so that the support frame 130 can be electrically connected to the washing and drying nozzle device 200.

The washing and drying nozzle device 200 includes: a drying nozzle 220 configured to dry a user's body by radiating far-infrared rays onto the user's body and spraying air at a predetermined pressure; a washing nozzle 250 configured to wash the user's body after defecation; a nozzle driving means 260 configured to perform driving so that the sliding movement and rotating operation of the drying nozzle 220 and the washing nozzle 250 can be performed; a sensor unit 310 configured to detect the presence of a foreign material on the surfaces of the drying nozzle 220 and the washing nozzle 250, and to keep the drying nozzle 220 and the washing nozzle 250 clean; and a control unit 300 configured to control the driving of the nozzle driving means 260 so that the user's body can be washed and dried after defecation, and to perform hygiene management on the surfaces of the drying nozzle 220 and the washing nozzle 250 according to information about the detection result transmitted from the sensor unit 310.

The drying nozzle 220 is connected to the nozzle driving means 260 and performs drying by discharging air to a user's body at a pressure set through the control unit 300. An air inlet hole 222 configured such that air is introduced thereinto at a predetermined pressure is formed in one side of the rear portion of the drying nozzle 200, and an air discharge hole 224 configured such that introduced air is discharged therethrough is formed in the top portion of the front portion of the drying nozzle 200.

The drying nozzle 220 is provided with a lamp assembly 228 formed under the air discharge hole 224 and configured to radiate far-infrared rays onto a user's body or the surface of the washing nozzle 250 through the air discharge hole 224 under the control of the control unit 300.

The lamp assembly 228 radiates far-infrared rays when air is sprayed onto a user's body wet due to washing after defecation, thereby further providing a sterilization function for the user's body and a sitz bath function through far-infrared rays. In other words, the lamp assembly 228 is configured to enable a fomentation function for a user's specific body part.

In addition, the drying nozzle 220 is provided with an operation support flange 226 configured to perform support so that an electrical connection between the lamp assembly 228 and the control unit 300 is made at the center of the rear side of the drying nozzle 220 and to also support the sliding movement and rotating operation of the drying nozzle 220. The operation support flange 226 accommodates the power cable of the lamp assembly 228 in the front thereof, supports the power cable so that it can be connected to the control unit 300, and is configured such that a connection with the nozzle driving means 260 is achieved in the rear of the operation support flange 226.

The operation support flange 226 is provided with a nozzle operation support means 240 configured to be connected to the end of the nozzle driving means 260 at its rear end thereof, to support a connection between the power cable and the control unit 300, and to transfer the rotational force of the nozzle driving means 260 to the operation support flange 226 to perform a rotation operation for the up and down reversal of the drying nozzle 220.

In this case, the nozzle operation support means 240 is configured to slide along with the drying nozzle 220 when the drying nozzle 220 is made to slide forward or backward by the nozzle driving means 260.

Meanwhile, the air supply member 230 is provided with a nozzle coupling portion 232 configured to be inserted over the drying nozzle 220 in order to enable the rotation of the drying nozzle 220 in one side thereof, and an air injection hole 234 configured to communicate with the air inlet hole 222 formed in the drying nozzle 220 and inject a predetermined amount of air is formed in the other side of the inner peripheral surface of the nozzle coupling portion 232.

In addition, the air supply member 230 is configured to be connected to an air supply device mounted inside the support frame 130, and is provided with an air supply portion 236 configured to supply the air, supplied from the air supply device, toward the air injection hole 234.

The drying nozzle 220 is configured to be rotated at intervals of 180° when the nozzle driving means 260 is driven. When the drying nozzle 220 is rotated, the air discharge hole 224 is moved downward and, at the same time, the air inlet hole 222 communicates with the air injection hole 234 of the air supply member 230, so that a predetermined amount of air is injected. Thereafter, when the drying nozzle 220 is rotated again at an interval of 180°, the communication between the air inlet hole 222 and the air injection hole 234 is released and, at the same time, the air discharge hole 224 is located below the drying nozzle 220, so that the discharge of air can be stopped.

Furthermore, the drying nozzle 220 may be configured such that the air discharge hole 224 is located below the washing nozzle 250 while the upward rotation operation is performed under the control of the control unit 300. In this case, the control unit 300 performs control so that the supply of air and the driving of the lamp assembly 228 are performed, with the result that the washing nozzle 250 configured to be spaced apart from the top of the drying nozzle 220 can be sterilized.

In other words, according to the present invention, the driving of the drying nozzle driving means 262 of the nozzle driving means 260 is controlled under the control of the control unit 300 when the drying function is performed after the completion of the washing function, and also the operation of the lamp assembly 228 is controlled according to the direction of the air discharge hole 224.

In addition, the washing nozzle 250 is configured to receive and discharge a predetermined amount of water so that the washing process is performed. One water pressure discharge hole 252 is formed in the bottom of the washing nozzle 250, and is configured to discharge supplied water at a predetermined pressure so that the water is sprayed toward a user's body.

In this case, the washing nozzle 250 may be coupled to the support frame 130 to be selectively drawn into and out of the support frame 130, and is connected to the washing nozzle driving means 264 of the nozzle driving means 260 to slide and rotate.

The washing nozzle 250 is coupled to the water supply unit 256 connected to the support frame 130, and is configured to spray water supplied from the water supply unit 256. Preferably, the washing nozzle 250 is configured to be disposed under the drying nozzle 220 that is drawn into and out of the support frame 130 and is also rotated at a predetermined angle.

In this case, the washing nozzle 250 is configured to operate preferentially after defecation under the control of the control unit 300. Preferably, the washing nozzle 250 is configured to be drawn out of the support frame 130 and spray a predetermined amount of water, and to be drawn into the support frame 130 when washing is completed. Thereafter, the drying nozzle 220 is operated under the control of the control unit 300.

The washing nozzle 250 of the present invention may be configured to spray a predetermined amount of water while being driven independently of the drying nozzle 220 under the control of the control unit 300.

However, the washing nozzle 250 of the present invention is not limited thereto, but may be further provided with a nozzle connection means 254 at the rear thereof to be connected to the washing nozzle driving means 264 and be configured to receive a predetermined amount of water and move toward the water pressure discharge hole 252.

In other words, the nozzle connection means 254 is configured to support the sliding movement of the washing nozzle 250 by connecting the rear end of the washing nozzle 250 and the front end of the washing nozzle driving means 264, and to allow rotating operation at a predetermined angle by transferring the rotational force, provided from the driving means 264, toward the washing nozzle 250.

Furthermore, the nozzle connection means 254 is configured to supply a predetermined amount of water to the rear end of the washing nozzle 250. Preferably, the nozzle connection means 254 is connected to the bottom surface of the water supply unit 256, and is connected to allow sliding movement from the water supply unit 256 so that the washing nozzle 250 can slide and supply water stably.

The washing nozzle 250 of the present invention configured as described above may be configured to be combined with the drying nozzle 220 in various ways according to the shape of the toilet seat-cover device 100.

As an example, the washing nozzle 250 of the present invention may be formed inside the drying nozzle 220 and spaced apart from the top surface of the lamp assembly 228. The end of the front portion of the washing nozzle 250 may be selectively drawn into and out of the drying nozzle 220 while sliding in the forward or backward direction of the drying nozzle 220. In other words, the washing nozzle 250 and the drying nozzle 220 may be integrated with each other.

The control unit 300 controls whether to drive the drying nozzle 220 and the washing nozzle 250. The control unit 300 controls whether to drive the nozzle driving means 260 so that the sliding movement and rotating operation of the drying nozzle 220 and the washing nozzle 250 can be controlled.

The control unit 300 controls the drying nozzle 220 and the washing nozzle 250 so that they can be driven independently of each other.

In addition, the control unit 300 further includes a sensor unit 310 configured to determine whether a foreign material such as excrement is present on the surface of the washing nozzle 250.

The sensor unit 310 is formed on one side of the support frame 130. The sensor unit 310 is configured to detect whether a foreign material is present on the surface of the washing nozzle 250 configured to be drawn into and out of the support frame 130, and to transmit a detection result information to the control unit 300.

Although the sensor unit 310 has been described as being formed in the support frame 130, it is not limited thereto, but may be formed inside the drying nozzle 220 and detect whether a foreign material is present on the surface of the washing nozzle 250.

In this case, a configuration may be made such that when information about the detection result transmitted from the sensor unit 310 implies that the foreign material is present, the control unit 300 allows the washing nozzle 250 to be sterilized by operating the lamp assembly 228 formed in the drying nozzle 220.

In addition, the control unit 300 may be configured to control the driving of the drying nozzle 220 and the washing nozzle 250 according to whether the toilet seat-cover device 100 is used, so that sterilization can be performed around the toilet bowl even when not in use.

In this case, the control unit 300 controls the driving of the nozzle driving means 260 so that water can be sprayed around a toilet bowl by rotating the washing nozzle 250 over a predetermined angle in the left and right directions and then drying and sterilization can be performed by rotating the drying nozzle 220 in the left and right directions.

Moreover, the control unit 300 operates the lamp assembly 228 formed in the drying nozzle 220 even while a user is defecating, so that far-infrared rays can be radiated toward a user's body, thereby providing a fomentation effect.

Meanwhile, the control unit 300 of the present invention may further include a remote control configured to provide driving control signals including a control signal for the starting or stopping of the driving of the washing and drying nozzle device 200 and a control signal for washing and drying operations, control signals for whether to use hot water including a control signal for the temperature of water supplied during washing, control signals for whether to emit far-infrared rays including a control signal for the independent driving of the lamp assembly 228, and control signals for the physical condition of a user, such as a child, an old person, a woman, or the like. The control unit 300 may be configured to receive a control signal transmitted from the remote control and control the driving of the washing and drying nozzle device 200.

According to an embodiment of the present invention, there is provided the washing and drying nozzle device for a sanitary toilet seat cover that radiates far-infrared rays directly to a user's body, so that effects can be achieved in that drying efficiency can be improved after washing with water and a sitz bath function and a sterilization function can be provided by the efficacy of far-infrared rays, thereby enabling sanitary management after the defecation of the user and health management for a specific body part.

Furthermore, according to an embodiment of the present invention, there is provided the washing and drying nozzle device for a sanitary toilet seat cover that radiates far-infrared rays toward the water pressure nozzle before, during, and after the use of a bidet, so that effects can be achieved in that the water pressure nozzle can be sterilized regardless of whether the bidet is used, thereby enabling the sanitary management of the water pressure nozzle.

Moreover, according to an embodiment of the present invention, there is provided the washing and drying nozzle device for a sanitary toilet seat cover that provides water and far-infrared rays to a user by using the water pressure nozzle and the drying nozzle, so that it is possible to dispose of defecation sanitarily even without using toilet paper, with the result that effects can be achieved in that the present invention is eco-friendly, can improve toilet culture, and can be used conveniently by the elderly, patients, children, and the disabled.

In addition, various advantageous advantages and effects of the present invention are not limited to the above-described effects, and will be more easily understood in the process of describing specific embodiments of the present invention.

The above description is merely an example of the technical spirit of the present invention, and various modifications and variations may be made by those of ordinary skill in the art without departing from the essential characteristics of the present invention. Accordingly, the embodiments disclosed in the present invention are not intended to limit the technical spirit of the present invention, but are intended to describe the technical spirit of the present invention. The scope of the technical spirit of the present invention is not limited by these embodiments. The range of protection of the present invention should be construed based on the attached claims, and all technical sprits within the equivalent range should be construed as being included in the scope of the present invention.

What is claimed is:

1. A washing and drying nozzle device for a sanitary toilet seat cover, the washing and drying nozzle device comprising:
a toilet seat-cover device comprising a support frame configured such that a cover member and a seat are rotatably coupled thereto and a sensor unit configured to detect presence of a foreign material is mounted on one side of a front portion thereof;
a drying nozzle coupled to the support frame, and configured to dry a user's washed body by spraying air having a predetermined pressure toward the user's body and to radiate far-infrared rays toward the user's body;
a washing nozzle coupled to the support frame, and configured to spray a predetermined amount of water toward the user's body;
a nozzle driving means installed in the support frame, and configured to slide and rotate the drying nozzle and the washing nozzle; and
a control unit configured to control driving of the nozzle driving means;
wherein the control unit receives detection result information about whether a foreign material is present on a surface of the washing nozzle from the sensor unit, and, when a foreign material is present, drives the drying nozzle to radiate far-infrared rays toward the washing nozzle so that sterilization is achieved.

2. The washing and drying nozzle device of claim 1, wherein the drying nozzle comprises:
an air inlet hole formed in one side of a rear portion of the drying nozzle, and configured such that air having a predetermined pressure is introduced therethrough;
an air discharge hole formed in a top of a front side of the drying nozzle, and configured such that introduced air and far-infrared rays are discharged therethrough;
a lamp assembly disposed below the air discharge hole, and configured to radiate far-infrared rays onto the user's body or the surface of the washing nozzle through the air discharge hole under control of the control unit;
an operation support flange disposed at a center of a rear side of the drying nozzle, and configured to perform support so that an electrical connection is made between the lamp assembly and the control unit and to support sliding movement and rotation operation of the drying nozzle;
a nozzle operation support means configured to connect the operating support flange and the nozzle driving means to each other, and to transmit a rotational force of the nozzle driving means to the operating support flange so that a rotating operation for up/down inversion of the drying nozzle can be performed; and
an air supply member configured to supply a predetermined amount of air toward the air inlet hole.

3. The washing and drying nozzle device of claim 1, wherein the washing nozzle is independently disposed below the drying nozzle, or is coupled to allow sliding movement inside the drying nozzle.

4. The washing and drying nozzle device of claim 1, wherein the washing nozzle further comprises a nozzle connection means configured to support a sliding movement and rotating operation of the washing nozzle and to supply a predetermined amount of water.

5. The washing and drying nozzle device of claim 1, wherein the control unit controls driving of the nozzle driving means so that water can be sprayed around a toilet bowl by rotating the washing nozzle over a predetermined angle in left and right directions and then drying and sterilization can be performed by rotating the drying nozzle in left and right directions.

6. The washing and drying nozzle device of claim 1, wherein the control unit performs control so that far-infrared rays can be radiated toward the user's body by operating the drying nozzle even while the user is defecating.

7. The washing and drying nozzle device of claim 1, wherein the control unit operates in conjunction with a remote control that provides a drive control signal, a control signal for whether to use hot water, a control signal for whether to radiate far-infrared rays, control signals for washing and drying operations, and a control signal for the user's physical condition.
